(19) United States
(12) Patent Application Publication
(10) Pub. No.: US 2005/0247121 A1
(43) Pub. Date: Nov. 10, 2005

(54) Urine collection and monitoring system

(75) Inventor: Michael Pelster, Warstein-Hirschberg (DE)

Correspondence Address: THE FIRM OF KARL F ROSS, 5676 RIVERDALE AVENUE, PO BOX 900, RIVERDALE (BRONX), NY, 10471-0900, US

(73) Assignee: Transmed Medizintechnik GmbH & Co. KG

(21) Appl. No.: 11/126,384

(22) Filed: May 6, 2005

(30) Foreign Application Priority Data
May 7, 2004 (EP) 04010870.6

(51) Int. Cl. G01F 11/28; A61B 5/20

(52) U.S. Cl. 73/223; 600/573; 600/581; 600/584

(57) ABSTRACT

A liquid-collection and -measuring system has a laterally and downwardly closed collection vessel, an input line for feeding liquid into the vessel, and a collection chamber horizontally generally level with the collection vessel. An upright dip tube has an open lower end in a lower region of the vessel and an upper end connected to tubing extending to the collection chamber. A pump along the tubing can advance liquid from the dip tube to the collection chamber.

### FIELD OF THE INVENTION

The present invention relates to a flow-measuring system. More particularly this invention concerns a urine-collecting and measuring system.

### BACKGROUND OF THE INVENTION

When a patient is catheterized it is standard to monitor his or her urine output. The traditional method is for the nursing staff to check the patient hourly, determine how much urine has been emitted since the last check, and make a note of it on the patient's chart. This can be done most accurately by having the urine from the catheter flow into a graduated collection vessel so the nurse can easily determine exactly how much has been emitted, and once this is determined the collection vessel is emptied into a larger container, typically a pouch, if it needs to be retained, e.g. for testing, or is discarded.

More complex automatic systems have been proposed which determine the amount of urine outputted by capacitative measurement or weighing. Weighing the urine is a problem, as the density of urine can vary, typically between 1.002 to 1.06 g/ml, particularly when the urine contains blood or fat. Thus the actual weight of the outputted urine can provide data that is less useful than knowing the volume outputted.

Another problem is that urine is not outputted continuously. In addition it is possible that there is considerable tubing between the patient and the collection chamber and this tubing extends mainly horizontally. In a system with tubing having an inside diameter of 7 mm and a length of 1.2 m, it is possible for 46 ml of urine to pool in the tubing so that, when the patient moves, all this liquid suddenly flows into the collection vessel, which itself is often attached to the bed so that, when the bed's inclination is changed, the collection vessel is tipped. These factors all lead to inaccurate measurements and occasional leaks or spills.

A solution using a pump is described in German 3,411,449 of Lück. Here a peristaltic pump is provided along the tubing running from the catheter to the collection bag. A sensor upstream along the tubing from the pump detects the presence of liquid in the tubing at this location and runs the pump so long as it detects liquid. Since the pump is of the positive-displacement type that moves a predetermined volume of liquid with each revolution, pump rotation can be monitored to get an accurate measurement of the liquid passing through the system.

Such a system is relatively bulky, as there must be a certain amount of tubing above the input to the pump and below the measurement location, acting as a reservoir. Thus when the device is hung on the patient's bed, it reaches all the way to the floor, putting it in danger of being kicked or damaged, and making it generally inconvenient to use. Furthermore the sensor is typically directly exposed to the liquid and with time is corroded or fouled, and under any circumstances must be discarded after use. Even during a single use, a salt coating from the urine can change the sensitivity of the sensor, for instance by increasing its resistance, so as to impede proper operation or cause the device to malfunction.

A similar system is described in U.S. Pat. No. 5,532,936 of LeVeen the catheter feeds urine into the top of a column whose lower end is connected via a tube to the collection pouch. A peristaltic pump along the tube is turned on and off by sensors at the top and bottom of the column. This arrangement is relatively effective, but once again takes up a great deal of physical space, with the pump below the column and the tubing following a circuitous path from the column to the collection pouch. The lengthy tubing is a particular problem as it greatly increases the possibility of accidents and spills.

### OBJECTS OF THE INVENTION

It is therefore an object of the present invention to provide an improved flow-measuring system.

Another object is the provision of such an improved flow-measuring system that overcomes the above-given disadvantages, in particular that is usable as a urine-collection device.

### SUMMARY OF THE INVENTION

A liquid-collection and -measuring system has according to the invention a laterally and downwardly closed collection vessel, an input line for feeding liquid into the vessel, and a collection chamber horizontally generally level with the collection vessel. An upright dip tube has an open lower end in a lower region of the vessel and an upper end connected to tubing extending to the collection chamber. A pump along the tubing can advance liquid from the dip tube to the collection chamber.

The collection vessel thus does not need to be above the collection chamber, so that the entire system can easily be hung, for instance, on the side of the bed of a catheterized patient. The system is fairly wide, but normally there is ample horizontal room.

The dip tube is rigid and is fixed in the vessel. This makes it possible for the tubing to be fairly short so that the likelihood of it getting crimped or damaged is minimized. The long reach of tubing from the bottom of a collecting column up to the top of the collection chamber is eliminated.

This construction gives several advantages. All the connections are in the upper region of the collection vessel, making it fairly easy to connect to it and hook up the collection system. In fact the collection vessel can fit in a socket of support carrying the pump for very quick setup.

In addition the connections to the collection vessel are simple and difficult to get wrong. Preferably the collection vessel is mounted on the support relative to the pump such that the upper end of the dip tube is directed right into the pump, making it unlikely that the connection with the incoming catheter will be confused with the connection with the tubing going through the pump to the collection chamber, which normally is a pouch. Setting up such a system is extremely simple and is virtually impossible to get wrong.

The tubing according to the invention is elastically deformable and the pump is a peristaltic pump. In addition the vessel has an upper region above the lower region and having a cross-sectional size substantially greater than a cross-sectional size of the lower region.

In accordance with the invention a sensor outside the vessel at an upper end of the lower region is capable of detecting liquid in the vessel, and a controller connected between the sensor and the pump operates the pump when the sensor detects liquid filling the lower vessel region. This sensor optically detects the liquid in the vessel. In fact two such sensors can be provided, a lower one that turns off the pump and an upper one that turns it on. This upper one is also run once an hour to empty the collection vessel down to the lower sensor so as to give an accurate measurement of liquid received in the hour.

To prevent backup in case of equipment failure an overflow line extends from an upper region of the vessel to the collection chamber.

### BRIEF DESCRIPTION OF THE DRAWING

The above and other objects, features, and advantages will become more readily apparent from the following description, reference being made to the accompanying drawing in which:

FIG. 1 is a largely schematic view of the flow-measuring apparatus according to the invention; and

FIG. 2 is an end view of a detail of FIG. 1.

### SPECIFIC DESCRIPTION

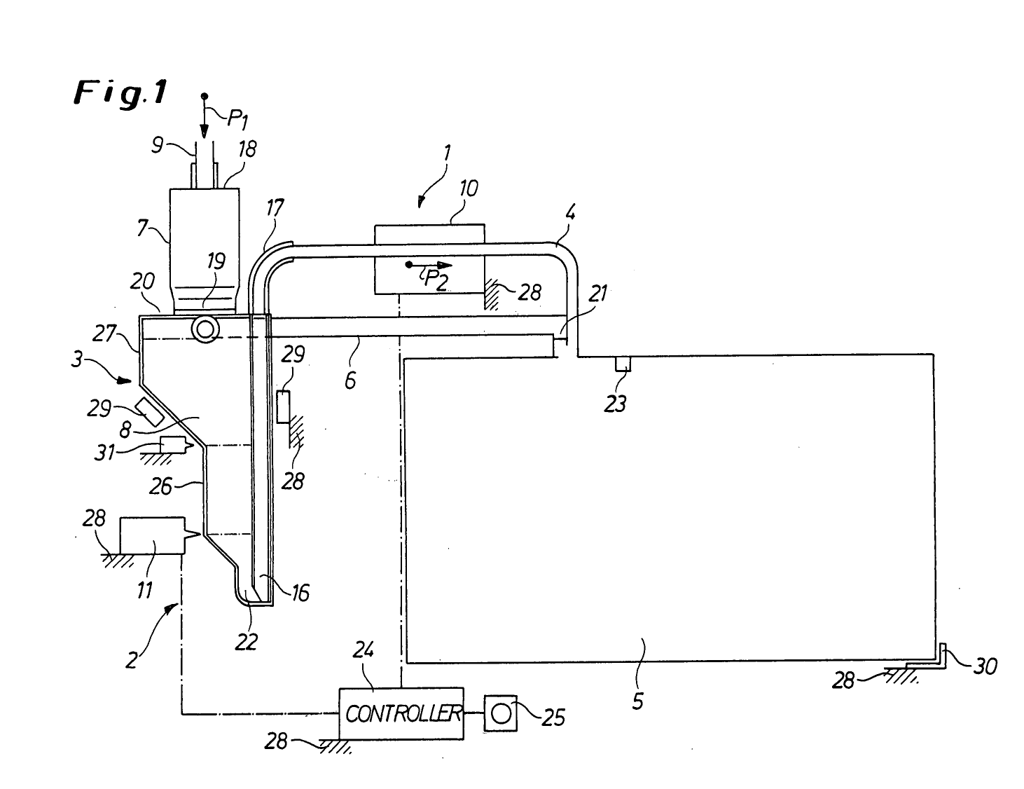

As seen in FIG. 1 a urine-collection system basically comprises a single-use disposable collection assembly 2 and a reusable control assembly 1.

The collection assembly 2 has a collector 3 comprised of a downwardly and laterally closed collection vessel or cup 8 and a drip cup 7. The vessel 8 has a lower end region 22 of very small cross section, an intermediate region 26 of larger cross section, and an upper end region 27 of relatively large cross section. A Foley catheter 9 or the like empties through a vent membrane 18 into the top of the drip cup 7 which in turn empties via a check valve 19 and vent membrane 20 into the top of the cup 8.

A straight dip tube 16 has a lower end fitted in the lower end region 22 of the cup 8 and an upper end fitted to or formed as a right-angle elbow 17 to which is fitted flexible tubing 4 passing through a pump 10 of the control assembly 1. This tubing 4 feeds into a large collection chamber formed by a pouch or bag 5 having an air vent 23 and forming the rest of the single-use collection assembly 2. An overflow tube 6 has one end opening into the upper region 27 of the cup 8 and an opposite end opening via a one-way valve 21 into the pouch 5. The tubing 4 can be of high-quality silicone with platinum and a polytetrafluoroethylene lining so that it can be repeatedly crushed without damage and will not lime up internally. Since the dip tube 16 is formed integrally with the cup 8, only a short length of this expensive tubing 4 is needed.

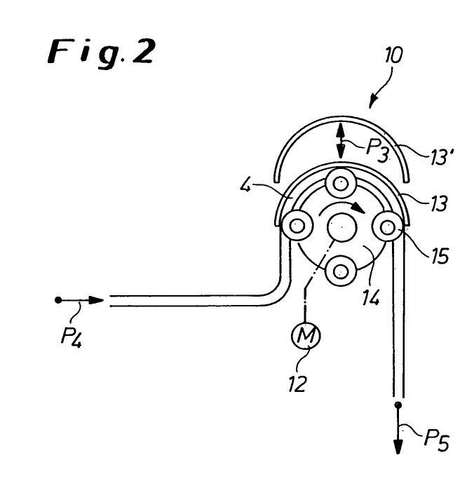

The pump 10 as shown in FIG. 2 is of the peristaltic type having a rotor 14 carrying four rollers 15 that compress the tubing 4 against an arcuate abutment plate 13 so as to take in liquid as shown by arrow P₄ and output it as shown by arrow P₅. A servomotor 12 steps the rotor 14 through 1800 increments and stops in end positions in which the tubing 4 is squeezed shut at three locations, preventing loss of prime in the tubing 4. The abutment plate 13 is part cylindrical and can be moved toward and away from the axis of the rotor 14 as shown by arrow P₃ to the position shown at 13′ to allow the tubing 4 to be taken out of the pump 4. With this system each half revolution of the rotor 14 moves 0.90 ml in a flow direction P₂ through the pump 10.

The control assembly 1 further comprises a microprocessor-type controller 24 connected to a drive motor 12 for the pump 10, a sensor 11 aligned between the lower and middle regions 22 of the cup 8, and a display 25. The sensor 11 is a light curtain or the like and is wholly outside the cup 8, which is made of transparent plastic. It is capable of detecting the liquid level inside the cup 8, for instance by projecting a laser-light beam of a wave length equal to the resonant frequency of water, which is the main constituent of urine, so that it can detect when the level in the cup 8 rises above the lower region 22 when the light beam does not pass through the cup 8 to a photocell on the opposite side. Here a quantity equal to 3 ml of liquid will fill the cup 8 enough to trip the lower sensor 11, and 20 ml to trip an upper sensor 31. The cup 8 holds 20 ml of liquid when the regions 22 and 26 are full, and can hold 80 ml before the liquid level reaches the overflow tube 6. The second sensor 31 is provided at the top of the intermediate region 26.

The pump 10, controller 24, and sensors 11 and 31 are all fixedly mounted on a common support indicated schematically at 28. In addition the vessel 8, which typically is integrally formed with the dip tube 16, connecting elbow 17, and cup 7 is fitted in an upwardly open socket 29 fixed to the support 28. The pump 10 is provided slightly above the vessel 3 and chamber 5 and in direct horizontal alignment with the horizontally open end of the elbow 17 so that the tubing 4 can pass straight from the pump 10 to this elbow 17. The pouch forming the collection chamber 5 is held in a seat or holder 30 on the support 28 horizontally adjacent the cup 3.

In use urine or any other liquid whose flow needs to be monitored and measured arrives via the catheter 9 as shown by arrow P₁ and drips into the cup 7, which primarily serves to eliminate bubbles and make the flow fairly uniform. Thence the liquid passes into the cup 8 which it fills from the bottom up. The open lower end of the dip tube 16 is in the lowermost quarter of the lower region 22 so that it is quickly completely submerged.

When the liquid level reaches the sensor 31, it sends a signal output to the controller 24 to energize the motor 12 and run the pump 10 until the level is below the sensor 11. In addition every hour the pump 10 is run to drain the cup 8 down to the level of the sensor 11, leaving only 3 ml in the cup 8, so that the total urine output in the hour is accurately determined. The downwardly decreasing cross-sectional size of the cup 8 insures that the pump 10 can easily keep up with any normally encountered input flow. If there is some accident or equipment malfunction, the overflow or bypass line 6 will shunt excess liquid past the pump 10 into the large collection pouch 5. Since the pump 10 is a constant-displacement unit it is possible for the controller 24 to easily calculate the amount of liquid moved, as it is directly proportional to the number of revolutions of the rotor 14, which is in turn controlled by the servomotor 12. On startup with the tubing 4 empty upstream of is the pump 10 there is admittedly some error as the pump 10 sucks liquid up into and fills the tubing 4, but this is minor so that the initial measurement can be disregarded.

The controller 24 calculates the amount of liquid moved by the pump 10 simply by multiplying the half-revolution pumped volume, here 0.90 ml by the number of half revolutions. The results can be displayed and held in memory, or can even be printed out on a sticker that can be affixed to the patient's chart.

The shape of the cup 8 is such that it can be tipped as much as 45° to the horizontal without leaking or causing the system to malfunction. Thus the support 28 can even be mounted on an end of the bed that is raised or inclined, but will still work properly.

1. A liquid-collection and -measuring system comprising:
a laterally and downwardly closed collection vessel having a lower region;
means including an input line for feeding liquid into the vessel;
a collection chamber horizontally generally level with the collection vessel;
an upright dip tube having an open lower end in the lower region of the vessel and an upper end;
tubing extending between the dip-tube upper end and the collection chamber; and
a pump along the tubing operable to advance liquid from the dip tube to the collection chamber.

2. The collection and measuring system defined in claim 1 wherein the dip tube is rigid and is fixed in the vessel.

3. The collection and measuring system defined in claim 2 wherein the tubing is elastically deformable and the pump is a peristaltic pump.0

4. The collection and measuring system defined in claim 3 wherein the vessel has an upper region above the lower region, the upper region having a cross-sectional size substantially greater than a cross-sectional size of the lower region.

5. The collection and measuring system defined in claim 3, further comprising
a sensor outside the vessel at an upper end of the lower region and capable of detecting liquid in the vessel; and
control means connected between the sensor and the pump for operating the pump when the sensor detects liquid filling the lower vessel region.

6. The collection and measuring system defined in claim 5 wherein the sensor optically detects the liquid in the vessel.

7. The collection and measuring system defined in claim 3, further comprising
an overflow line extending from an upper region of the vessel to the chamber.
